(19) United States
(12) Patent Application Publication
(10) Pub. No.: US 2023/0313260 A1
(43) Pub. Date: Oct. 5, 2023

(54) SELECTIVE MEDIA FOR DETECTION OF ZYGOSACCHAROMYCES

(71) Applicant: Plant Response, Inc., Redmond, WA (US)

(72) Inventor: Lance Jacobe, Redmond, WA (US)

(73) Assignee: Plant Response, Inc., Redmond, WA (US)

(21) Appl. No.: 17/996,709

(22) PCT Filed: Apr. 5, 2021
(86) PCT No.: PCT/US2021/025795

§ 371 Date: Oct. 20, 2022

Related U.S. Application Data
(60) Provisional application No. 63/013,961, filed on Apr. 22, 2020.

(51) Int. Cl. C12Q 1/04

(52) U.S. Cl. CPC C12Q 1/04; C12Q 1/045

(57) ABSTRACT

Selective media for detection and quantification of Zygosaccharomyces yeast and methods of their preparation and use are disclosed.

### CROSS REFERENCE TO RELATED APPLICATION

The present application claims priority to the U.S. Provisional application number 63/013,961 filed on Apr. 22, 2020 and entitled “SELECTIVE MEDIA FOR DETECTION OF ZYGOSACCHAROMYCES,” the disclosure of which is hereby incorporated by reference in its entirety.

### TECHNICAL FIELD

The disclosed embodiments relate to a selective medium for growth, detection and quantification of Zygosaccharomyces yeasts and methods of its preparation and use.

### BACKGROUND

Organic liquid fertilizers can be prepared using technologies to extract and stabilize nutrients derived from non-putrefied food waste. Inherent to food waste are both pathogenic and nuisance/spoilage microorganisms. While materials are processed to circumvent spoilage and growth of pathogenic microorganisms, under natural environmental conditions certain organisms may persist. One such organism is Zygosaccharomyces bailii (Zb). While non-pathogenic, Zb is a common spoilage organism in products across food industries including wine, beer, organic fertilizers, juice, and jams.

Not only is Zb resistant to most food preservatives, but when environmental conditions become unfavorable, Zb will sexually reproduce creating ascospores. These spores are notoriously hard to kill as they share the same preservative resistance but can now survive in up to 60° C. for up to 15 minutes before viability is affected. While in the vegetative state, Zb also produces copious amounts of gas under a wide range of environmental conditions. Without vented packaging, gas buildup can cause bloating or rupture of containers, causing product recalls at great cost to the manufacturer. Consequently, a means of rapid Zb detection and quantification is required to determine the storage stability of shelf-stable products.

While Zb can grow on traditional microbial media, indeterminant background microbial growth prohibits consistent and accurate results. Thus, there is a need for a selective media necessary for adequate detection and quantification of Zygosaccharomyces bailii.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates an example selective pressure regime for selective growth of Zygosaccharomyces, in accordance with embodiments of the present disclosure;

FIG. 2 illustrates an example method for preparing a growth medium for selective growth of Zygosaccharomyces, in accordance with embodiments of the present disclosure; and

FIG. 3 illustrates an example method for selective growth of Zygosaccharomyces, in accordance with embodiments of the present disclosure.

### DETAILED DESCRIPTION

Spoilage organisms may exhibit resilience and/or resistance to processes for reducing organism viability in liquid media such as pasteurization, addition of chemical preservatives, osmotic pressure treatment, or pressure-driven cell wall disruption. Zygosaccharomyces bailii (Zb) is an example organism that is resistant to preservatives used in food and beverage products, byproducts, and waste. Zygosaccharomyces rouxii (Zr) is another example organism exhibiting similar, albeit different, resistance. While non-pathogenic, Zb and Zr are common spoilage organisms in process streams across food industries including wine, beer, organic fertilizers, juice, and jams. Not only are Zb and Zr resistant to most food preservatives, but when environmental conditions become unfavorable to vegetative growth (“budding”), Zb will sporulate. The spores of Zb present a significant challenge for denaturation, being stable at temperatures as high as 60° C. for up to 15 minutes before viability is affected. For at least this reason, process yield, product quality, and shelf stability benefit from an assay to identify the presence of Zb or Zr that is based on selective cultivation of Zb or Zr.

While being preservative-resistant and thermally-resilient, Zb and Zr can be out-competed by other microorganisms commonly found in food industry processes. In this way, an assay to identify the presence of Zb and/or Zr may be confounded by competitive growth of the other organisms. To selectively grow Zygosaccharomyces species, such as Zb, a growth medium can impose multiple selective pressures that, in combined effect, suppress the competitive growth of non-Zygosaccharomyces species, and thereby provide for improved detection and quantification. That being said, excess application of selective pressures can reduce or substantially eliminate the growth rate of Zygosaccharomyces species, introducing lead time into production processes to account for quality assurance protocols. For example, applying excessive pressure may result in Zb incubation periods of as much as a week, as much as two weeks, or more.

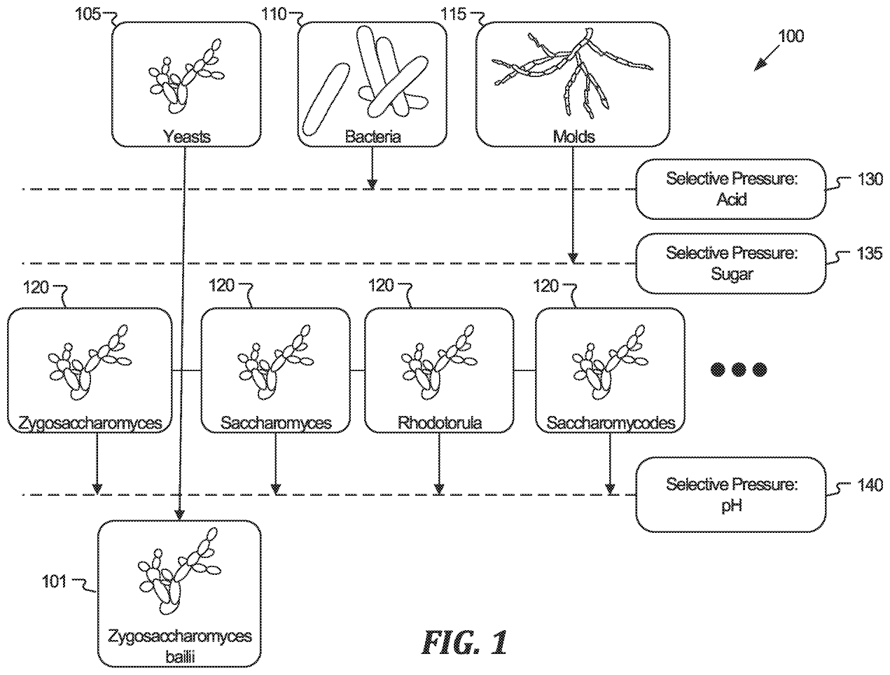

FIG. 1 illustrates an example selective pressure regime 100 for selective growth of Zygosaccharomyces bailii 101, in accordance with embodiments of the present disclosure. Methods and media are provided for an assay to selectively identify and quantify Zygosaccharomyces species in samples, the assay characterized by a reduced incubation time relative to existing materials and methods. Examples are provided describing selective growth and identification of Zygosaccharomyces bailii (Zb) 101, but embodiments of the present disclosure are not limited to a single species. A growth medium for selective growth and detection of Zb 101 may be applied to other species or genera of microorganisms that act as spoilage organisms in process streams of food, beverage, or agricultural products or byproducts. Viruses and parasites may also act as spoilage organisms, but are typically characterized by more complicated replication cycles (e.g., through infection of a host organism) and, as such, are detected with other techniques, since multiplication or spoilage will not occur in materials that can act only as a passive vehicle in the transmission of infection.

The diverse components of the growth medium serve to promote selective growth of Zb 101 over other competing microorganisms in a sample of a food, beverage, or agricultural process stream, product, or byproduct. For example, a growth medium may be formulated to implement the selective pressure regime 100, to be used with a sample including yeasts 105, bacteria 110, molds 115, among other microorganisms. The yeasts 105 may include multiple yeasts 120 from genera and/or species including, but not limited to, Zygosaccharomyces, Saccharomyces, Thodotorula, or Saccharomycodes. As part of the selective pressure regime 100, constituents of the growth medium may suppress the growth of at least a portion of the microorganisms, such that Zb 101 may grow with significantly reduced competition. In some embodiments, the selective pressure regime 100 includes acid 130, sugar 135, and pH adjustment 140 to suppress the competing microorganisms. While FIG. 1 illustrates the selective pressures in an exemplary order, the illustration is intended merely to simplify the explanation of the selective pressure regime 100, rather than to specify a particular order of action or a causal relationship between the selective pressures 130-140 and the selective growth of Zb 101. For example, growth of at least some of the yeasts 120 may be suppressed by the acid 130 alone, by the acid 130 and/or by the sugar 135, or by the combined action of the acid 130, the sugar 135 and the pH 140. Similarly, some bateria 110 may be suppressed by hypertonic osmotic pressure applied by the sugar 135, and some molds may be suppressed by the acid 130. Furthermore, selective growth is understood to be a combined effect of the selective pressures 130-145 acting in concert, rather than individually.

In one aspect, the disclosure provides a growth medium selective for Zygosaccharomyces bailii comprising a nutrient base, a sugar, formic acid, and acetic acid. In some embodiments, the growth medium of the disclosure comprises a nutrient base, about 1%-25% w/v sugar, about 0.1%-0.3% v/v formic acid, about 0.2%-0.5% v/v acetic acid, and has a final pH of between about 2.5 to about 4. In some embodiments, the sugar comprises glucose or fructose.

In some embodiments, the nutrient base comprises peptone, and/or yeast extract. In some embodiments, the nutrient base comprises peptone at a final medium concentration of about 0.5%-2%, yeast extract at a final medium concentration of about 0.1%-1%, and a final medium concentration of about 1% to 3%.

As part of the selective pressure regime 100, the sugar 135 influences the osmotic pressure of the growth medium and can suppress the growth of some yeasts 105, bacteria 110, and/or molds 115. Selecting a concentration for the sugar 135 within the range of about 1%-25% weight/volume provides an osmotic pressure within a range that is hypertonic for molds 115, for example, while remaining isotonic for at least some of the yeasts 105, including Zb 101. Providing sugar 135 outside the range may impair the effectiveness of the selective pressure regime 100. For example, a sugar concentration less than 1% may permit at least some yeasts 105, bacteria 110, and/or molds 115 to outcompete the Zb 101, while a sugar concentration greater than 25% may result in a hypertonic environment that suppresses growth of the Zb 101. In some embodiments, the growth medium comprises sugar 135 with a concentration in the growth medium of about 5%-25%, about 7%-25%, about 10%-25%, about 12%-25%, about 15%-25%, about 17%-25%, about 20%-25%, about 5%-23%, about 7%-23%, about 10%-23%, about 12%-23%, about 15%-23%, about 17%-23%, about 20%-23%, about 7%-21%, about 10%-21%, about 12%-21%, about 15%-21%, about 17%-21%, or about 20%-21%, expressed as a percent weight/volume. In some embodiments, the concentration of the sugar 135 in the growth medium is about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, expressed as a percent weight/volume.

As part of the selective pressure regime 100, acid 130 can exert a toxic effect and suppress the growth of some yeasts 105, bacteria 110, and/or molds 115. The acid 130 may be or include one or more diverse acids included in the growth medium. In some embodiments, the acid 130 includes acetic acid and/or formic acid. Advantageously, despite being a weak acid, acetic acid serves as a preservative in food and beverage products, and is effective at suppressing the growth of microorganisms found in food, food waste, beverages, beverage waste, liquids, agricultural products, and/or agricultural byproducts. Without being bound to a particular mechanism of action, the toxic effect of acetic acid results mostly from acetic acid dissociation inside microbial cells, causing a decrease of intracellular pH and metabolic disturbance by the anion, among other deleterious effects. Formic acid is a stronger acid than acetic acid, and may be included to suppress growth of microorganisms, including, but not limited to, Escherichia bacteria.

Providing formic acid at a concentration within the range of about 0.1%-0.3% volume/volume provides a toxic environment for some bacteria 110 and/or molds 115, for example, while remaining viable for at least some of the yeasts 105, including Zb 101. Providing acid 130 outside the range may impair the effectiveness of the selective pressure region 100. For example, an acid concentration less than 0.1% v/v may permit at least some yeasts 105, bacteria 110, and/or molds 115 to outcompete the Zb 101, while an acid concentration greater than 0.3% v/v may result in a toxic environment that suppresses growth of the Zb 101. In some embodiments, the growth medium of the disclosure further comprises formic acid. In some embodiments, the concentration of formic acid in the growth medium of the disclosure is about 0.12%-0.3%, about 0.15%-0.3%, about 0.17%-0.3%, about 0.2%-0.3%, about 0.22%-0.3%, about 0.25%-0.3%, about 0.12%-0.27%, about 0.15%-0.27%, about 0.17%-0.27%, about 0.2%-0.27%, about 0.22%-0.27%, about 0.25%-0.27%, about 0.12%-0.25%, about 0.15%-0.25%, about 0.17%-0.25%, about 0.2%-0.25%, about 0.12%-0.22%, about 0.15%-0.22%, about 0.17%-0.22%, or about 0.2%-0.22%, expressed as a percent volume/volume. In some embodiments, the concentration of formic acid in the growth medium of the disclosure is about 0.12%, about 0.15%, about 0.17%, about 0.2%, about 0.22%, about 0.25%, about 0.27%, or about 0.3%, expressed as a percent volume/volume.

Similarly, providing acetic acid at a concentration within the range of about 0.2%-0.5% volume/volume provides a toxic environment for some yeasts 105, bacteria 110 and/or molds 115, while remaining viable for at least some of the yeasts 105, including Zb 101. Providing acetic acid outside the range may impair the effectiveness of the selective pressure regime 100. For example, an acetic acid concentration less than 0.2% v/v may permit at least some yeasts 105, bacteria 110, and/or molds 115 to outcompete the Zb 101, while an acetic acid concentration greater than 0.5% v/v may result in a toxic environment that suppresses growth of the Zb 101. The growth medium of the disclosure further comprises acetic acid. In some embodiments, the concentration of acetic acid in the growth medium of the disclosure is about 0.22%-0.5%, about 0.25%-0.5%, about 0.27%-0.5%, about 0.3%-0.5%, about 0.32%-0.5%, about 0.35%-0.5%, about 0.37%-0.5%, about 0.4-0.5%%, about 0.42%-0.5%, about 0.45%-0.5%, about 0.47%-0.5%, about 0.22%-0.45%, about 0.25%-0.45%, about 0.27%-0.45%, about 0.3%-0.45%, about 0.32%-0.45%, about 0.35%-0.45%, about 0.37%-0.45%, about 0.4%-0.45%%, about 0.42%-0.45%, about 0.22%-0.4%, about 0.25%-0.4%, about 0.27%-0.4%, about 0.3%-0.4%, about 0.32%-0.4%, about 0.35%-0.4%, about 0.37%-0.4%, about 0.22%-0.35%, about 0.25%-0.35%, about 0.27%-0.35%, about 3%-3.5%, about 3.2%-3.5%, about 0.22%-0.3%, about 0.25%-0.3%, about 0.27%-0.3%, about 0.22%-0.27%, or about 0.25%-0.27%, about 0.27%-0.3%, expressed as a percent volume/volume. In some embodiments, the concentration of acetic acid in the growth medium disclosed herein is about 0.2%, about 0.25%, about 0.27%, about 0.3%, about 0.32%, about 0.35%, about 0.37%, about 0.4%, about 0.42%, about 0.45%, about 0.47%, or about 0.5% expressed as a percent volume/volume.

As the toxic effect of the acid 130 may result at least in part from metabolic processes or dissociation internal to microorganisms, the pH 140 of the growth medium may be a parameter of the selective pressure regime 100 independent of the acid 130. For example, sodium hydroxide may be added to the growth medium to provide a final pH 140 within a range. In some embodiments, the effects of sugar 135 and pH 140 are coupled, at least in that the Zb 101 is capable of growth at a higher composition of sugar 135 in the growth medium when pH 140 is maintained within a range of values. In this way, pH 140 above the range may permit unselective growth of competing microorganisms, while pH 140 below the range may suppress all growth and render an assay ineffective over an industrially relevant period of time. In some embodiments, the growth medium has an acidic final pH. In some embodiments, the growth medium has a final pH between about 2.7 and 4, between about 3 and 4, between about 3.2 and 4, between about 3.5 and 4, between about 3.7 and 4, between about 2.7 and 3.7, between about 3 and 3.7, between about 3.2 and 3.7, between about 3.5 and 3.7, between about 2.7 and 3.5, between about 3 and 3.5, between about 3.2 and 3.5, between about 2.7 and 3.2, or between about 3 and 3.2. In some embodiments, the growth medium has a final pH between about 2.5, about 2.6, about 2.7, about 2.8, about 2.9, about 3, about 3.1, about 3.2, about 3.3, about 3.4, about 20 3.5, about 3.6, about 3.7, about 3.8, about 3.9, or about 4.

Providing a protein hydrolysate to the growth medium supplies amino acids, peptides and proteins, which act as a source for nitrogenous nutrients. A diverse variety of protein hydrolysates, also called peptones, may be used, including, but not limited to products of enzymatic digestion or acid hydrolysis of natural products, such as animal tissues, milk, plants or microbial cultures. Peptones can promote and sustain the growth of most common organisms. In this way, selecting a composition of peptones within a particular range may reinforce the selective pressure regime 100. For example, with a composition of peptone below the range, the growth medium may be nitrogen deficient. Conversely, with a composition of peptone above the range, the growth medium may provide nitrogen for competing microorganisms. Similarly, other nutrients, such as yeast extract, may be selected within ranges that permit timely and selective growth of Zb. As such, in some embodiments, the growth medium comprises about 0.5%-1.5% w/v peptone, about 0.25%-0.75% w/v yeast extract, about 1.5%-2.5% w/v agar, about 10%-25% w/v glucose, about 0.15%-0.25% v/v formic acid, about 0.22%-0.45% v/v acetic acid, and has a final pH of between about 3 to about 4. In some embodiments, the growth medium comprises about 1% w/v peptone, about 0.5% w/v yeast extract, about 2% w/v agar, about 15%-22% w/v glucose, about 0.2% v/v formic acid, about 0.25% v/v acetic acid, and has a final pH of between about 3 to about 4.

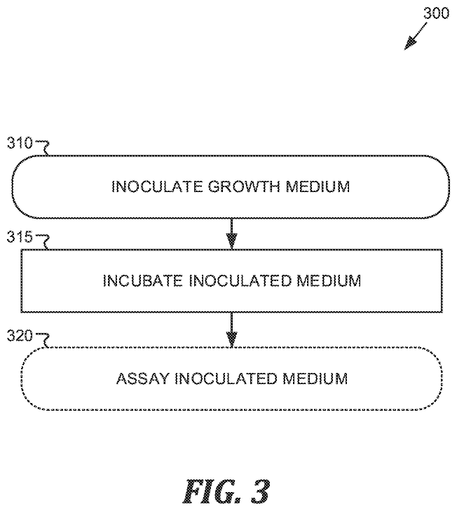

In some embodiments, the growth medium comprises further comprises a pH indicator suitable for use in microbiological growth media. In some embodiments, such indicator is bromocresol green. In some embodiments, the bromocresol green is provided at a concentration of from about 0.001% to about 0.005% w/v. The pH indicator included in the growth medium may facilitate quantification of selective growth, for example, by colorimetric means, as a result of acidification or alkalization of the intracellular environment during incubation, as also described in reference to FIG. 3, below. For example, Zb may ingest or otherwise absorb a pH sensitive dye that is distributed in the growth media. In this way, the acid concentration internal to the cells of Zb may shift the color of the pH sensitive dye internal to the cells, while leaving the color of the growth medium relatively unchanged. As growth of Zb continues and the acid 130 is depleted, Zb colonies may alkalize the acid 130 used by the microorganism as a carbon source during growth. In this way, the pH of the growth medium may increase over time and may induce a color change in the growth medium. Bromocresol green, for example, shifts from yellow to blue between pH 3.8 and pH 5.4. In this way, the number and size of Zb colonies may be identified and quantified as part of a selective growth assay using the growth medium described above.

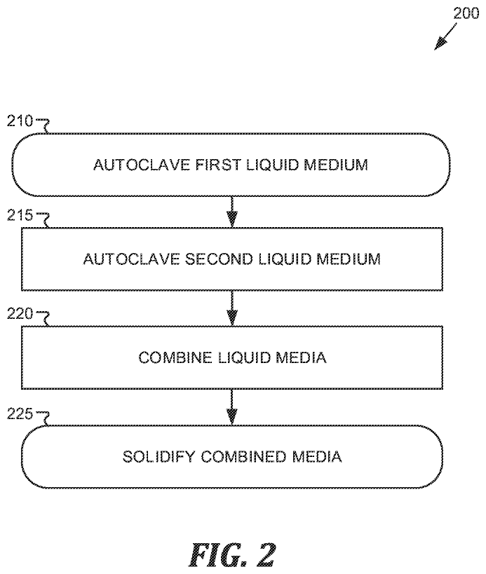

FIG. 2 illustrates an example method 200 for preparing a growth medium for selective growth of Zygosaccharomyces, in accordance with embodiments of the present disclosure. For example, the method 200 may describe a method of making a growth medium selective for Zygosaccharomyces bailii. The growth medium may be an example of growth medium 100 of FIG. 1. While the blocks of the method 200 are illustrated in an order, the operations described in the blocks need not be undertaken in a specific order. Some blocks may be optional, and some blocks may be omitted.

Block 210 illustrates autoclaving a first liquid medium comprising the formic acid, acetic acid, between about 30% and 70% of the total amount of glucose in the growth medium, and water. For the growth medium to be or include a gel, such as agar, while also providing an acid concentration suitable for the selective pressure regime as described in more detail in reference to FIG. 1, the acidic components of the growth medium may be provided in the first liquid medium. Advantageously, isolating the acidic components permits the autoclaving of the first liquid medium so

Block 215 illustrates autoclaving a second liquid medium comprising the peptone, yeast extract, agar, between about 30% and 70% of the total amount of glucose in the growth medium and water. As described in reference to block 210, the second liquid medium may include the agar, peptone, yeast extract, as well as other components of the growth medium that may be degraded by acid (e.g., acid 130 of FIG. 1) under high temperatures used for autoclaving.

Block 220 illustrates combining the first growth medium and second growth medium while still liquid, wherein the combination of the first growth medium and second growth medium comprises the total amount of glucose in the growth medium.

Block 225 illustrates allowing the combined growth medium to cool sufficiently to form a solid medium. Cooling the combined growth medium may include controlled cooling or ambient cooling, such that the growth medium forms a gel. In this way, the solid medium may describe an agar gel, where dissolved molecules may diffuse through the gel, while microorganisms are localized after inoculation.

FIG. 3 illustrates an example method 300 for selective growth of Zygosaccharomyces, in accordance with embodiments of the present disclosure. For example, the disclosure provides a method of selectively cultivating Zygosaccharomyces bailii. The method 300 for selective growth may employ the growth medium 100 of FIG. 1. As described in more detail in reference to FIG. 2, while the blocks of the method 300 are illustrated in an order, the operations described in the blocks need not be undertaken in a specific order. Some blocks may be optional, and some blocks may be omitted.

Block 310 illustrates inoculating a growth medium of the disclosure with a sample suspected of containing Z. bailii. Inoculation may describe one or more standard inoculation techniques, as would be understood by a person having ordinary skill in the art. For example, where the growth medium is provided as a gel plate, block 310 may optionally include sampling a surface using a sterile swab and depositing the sample on the gel plate.

In another example, where the growth medium is provided as a volumetric gel, block 310 may optionally include sampling a liquid (e.g., a beverage or liquid process stream of a food or agricultural process) and inoculating at least a portion of the liquid into the gel.

Block 315 illustrates incubating the inoculated medium under conditions sufficient to allow Z. bailii colony formation for at least 36 hours. In some embodiments, the method comprises incubating the inoculated medium at a temperature suitable for Zygosaccharomyces bailii growth, for example, at a temperature from about 25° C. to about 30° C. Optionally, block 315 may include incubating the inoculated medium for as long as about five days or longer, as an approach to selectively eliciting sporulation of Zb. Vegetative growth (budding) of Zb may be the dominant growth mechanism before three days (72 hours), while after five days (120 hours) growth may shift to favor sporulation. Spores of Zb present a particular challenge for spoilage of biological materials, at least because the spores of Zb are more resilient than Zb cells. Advantageously, as sporulation occurs through sexual reproduction of Zb, the method 300 permits the identification of viable colonies in a reduced incubation time, as compared to conventional characterization methods.

The methods of the disclosure, i.e., methods of selective cultivating Zygosaccharomyces bailii, can be used with any suitable sample. In some embodiments, the sample comprises or is derived from food, food waste, beverages, beverage waste, wine, agricultural products, and/or agricultural byproducts. In some embodiments, the sample is a test sample created from a surface that has contact with food, food waste, or agricultural products. In some embodiments, the methods can be used for testing of the presence of Zygosaccharomyces bailii on industrial food processing equipment, cooking and food prep surfaces, food containers, etc. The term “food” describes processed or unprocessed solid or semi-solid items produced for human or animal consumption. The term “food waste” describes portions of food that are discarded at one or more points in a food item supply chain (e.g., pre-consumer waste, post-consumer waste, etc.) or discarded food containers. The term “beverage” describes processed or unprocessed liquid items produced for human or animal consumption. The term “beverage waste” describes portions of beverages that are discarded at one or more points in a beverage item supply chain (e.g., pre-consumer waste, post-consumer waste, etc.) or discarded beverage containers. The term “wine” describes fermented alcoholic beverages derived from grapes or other fruit, waste derived from the wine-making process, waste derived from wine transport and distribution, and waste derived from wine consumption. The term “agricultural products” describes plant or animal material produced by agriculture, including, but not limited to material grown for human or animal consumption (e.g., fruit or vegetables), material grown for industrial refining (e.g., soy or canola), or material grown for agricultural purposes (e.g., cover matter or nitrification crops). The term “agricultural byproducts” describes waste and other material produced as part of the agricultural process, including, but not limited to, animal waste, discarded vegetable and fruit material, discarded animal parts, or the like.

Block 320 illustrates optionally assaying the inoculated medium to identify and/or quantify the Zb colony formation. Block 320 may be repeated multiple times during and/or after the incubation period of block 315. As such, the disclosure provides a method of detecting contamination of food, food waste, or agricultural products, or a surface in contact with food, food waste, or agricultural products, by Zygosaccharomyces bailii. The method may include inoculating a growth medium of the disclosure with a sample containing or derived from food, food waste, or agricultural products, or obtained from a surface in contact with food, food waste, or agricultural products, and incubating the inoculated medium under conditions sufficient to allow Z. bailii colony formation for at least 36 hours, and detecting formation of one or more Z. bailii colonies, indicating contamination.

In some embodiments, the method further comprises quantifying the one or more Z. bailii colonies, e.g., by methods known in the art. In some embodiments, the method further comprises comparing a level of quantified one or more Z. bailii colonies to a reference standard reflecting a known contamination level in a reference food, food waste, or agricultural product, or a reference surface in contact with food, food waste, or agricultural products. As described in reference to FIG. 2, quantification of Zb may include colorimetric techniques employing bromocresol green, or other pH sensitive dyes. By observing localized color change internal to microorganisms, the Zb colonies can be identified, and quantified, as expressed in terms of Colony Forming Units (CFU), used to estimate the number of viable Zb cells in a sample.

While each of the elements of the present disclosure is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature referred to herein are hereby incorporated by reference in their entirety as if each individual publication, patent or patent application were specifically and individually indicated to be incorporated by reference.

As used herein, “about” means plus or minus ten percent of the indicated value.

As can be appreciated from the disclosure above, the present invention has a wide variety of applications. The invention is further illustrated by the following examples, which are only illustrative and are not intended to limit the definition and scope of the invention in any way.

### EXAMPLES

A media that is minimally selective for Zygosaccharomyces bailii (Zb) consists of mineral media supplemented with vitamins and oligoelements, 0.005% bromocresol green, 0.4% formic acid, 0.1% glucose, and pH adjusted to 4.5. The bromocresol green indicator pigment results in Zb growth with a distinct color change due to the alkalization of the medium. Multiple pigments are available, and diverse pigments were experimentally assessed, but none provided the same observable color change as bromocresol green. Nutrient-restrictive selective medium also slowed growth of Zb to such an extent that identification was impossible due to overgrowth of background microbes. Thus, the use of the basal mineral media was discontinued as it lacked the nutrients to keep Zb alive under additional selective stresses that might be used to filter out unwanted microbial growth. Selective growth medium formulations were tested in multiple iterations according to a parametric design, for example, by varying one or more of the selective pressures described in reference to FIG. 1. Each iteration was left to incubate at 30° C. for 10 days to ensure accurate CFU recovery and growth timetable.

Formulaic changes between iterations are listed below in Table 1 and Table 2. Initial formulations were focused on the selectivity of formic acid and acetic acid. Both acids are used as preservatives in various industries but Zb can utilize it as a carbon source. Iteration 2 and 3 had 2% w/v peptone, 1% w/v yeast extract, 2% w/v glucose, and the pH was still too low for Zb growth. Further iterations had the nutrient base reduced by half to 1% w/v peptone, 0.5% w/v yeast extract, and 1% w/v glucose. Iterations 4 and 5 showed that adjusting the pH to approximately 3.5 permitted Zb growth, but the growth rate was too slow for rapid detection/quantification. Iterations 6 to 9 adjusted formic and acetic acid concentrations to favor Zb metabolism. These iterations retained selectivity but had little impact on metabolic rates.

Zb can thrive at a pH as low as 2.2, if sugar content is high enough. Thus, iterations 10 to 13 used a pH of 2.2 and 20% w/v glucose, for which growth time was reduced from 5 to 4 days. Table 2 shows iterations 14, 15, and 17 where the use of alternate preservative acids that Zb is known to be resistant to was attempted, but no growth was detected. Iteration 16 included formic acid, acetic acid, and glucose, a formulation which successfully reduced growth time of visible colonies to 48 hours but reduced overall recovery rates. Iterations 18 to 21 reduced acetic acid and varied the amount of glucose, with 18 being the most effective. It was found that 20% w/v glucose added to the selectivity by providing oxidative/osmotic stress to exclude background microbes while also allowing Zb to survive at the low pH. Vegetative cells were observable at 48 hours and those in a dormant spore state are visible between 72 to 96 hours. Additional experiments with fructose revealed that a 1:1 replacement of glucose with fructose provided substantially equal growth results, as measured by visible colony count.

TABLE 1
ITERATIONS OF ZB GROWTH MEDIA

| | Formic | Acetic | | | Growth |
|---|---|---|---|---|---|
| | acid | acid | Glucose | pH | (visible colony |
| Iteration | v/v | v/v | w/v | Adjusted | count) |
| 1 | 0.5% | 0.4% | 0.1% | no | no |
| 2 | 0.4% | 0.5% | 2% | no | no |
| 3 | 0.4% | 0% | 2% | no | no |
| 4 | 0.4% | 0.5% | 1% | yes, 3.54 | yes, 5 days |
| 5 | 0.4% | 0.5% | 1% | no | no |
| 6 | 0.4% | 0% | 1% | yes, 3.48 | yes, 5 days |
| 7 | 0.3% | 0.5% | 1% | yes, 3.47 | yes, 5 days |
| 8 | 0.2% | 0.5% | 1% | Yes, 3.47 | yes, 5 days |
| 9 | 0.4% | — | 1% | Yes, 3.47 | yes, 5 days |
| 10 | 0.4% | — | 6% | yes, 3.37 | yes, 5 days |
| 11 | 0.4% | 0.5% | 6% | yes, 3.47 | yes, 5 days |
| 12 | 0.4% | 0.5% | 21% | yes, 3.61 | yes, 4 days |
| 13 | 0.1% | — | 21% | yes, 3.68 | no inhibition of |
| | | | | | background |
| 16 | 0.2% | 0.5% | 21% | yes, 3.53 | Yes, 2 days |
| | | | | | reduced CFU |
| 18 | 0.2% | 0.25% | 21% | yes, 3.70 | Yes, 2 days |
| 19 | 0.2% | 0.25% | 11% | yes, 3.59 | yes, but less |
| | | | | | selective |
| 20 | 0.2% | 0.25% | 6% | yes, 3.59 | yes, but less |
| | | | | | selective |
| 21 | 0.2% | 0.25% | 1% | yes, 3.59 | yes, but less |
| | | | | | selective |

TABLE 2
ITERATIONS OF ZB GROWTH MEDIA

| | Formic | benzoic | Sorbic | Acetic | | |
|---|---|---|---|---|---|---|
| Iteration | acid | acid | acid | acid | Glucose | growth |
| 14 | 0.2% | 0.5% | — | — | 6% | no |
| 15 | — | 0.01% | 0.1% | 0% | 21% | no |
| 17 | — | 0.01% | 0.1% | 0.5% | 11% | no |

Two liquid media were prepared in Bottle 1 and Bottle 2 as shown in the table 3 below. Bottle 1 and Bottle 2 were autoclaved for 25 minutes on a liquid cycle. The components were combined while hot and poured within 30 min. It was found that the compositions of Bottle 1 and Bottle 2 must be autoclaved in two separate bottles. Low pH can hydrolyze agar and will prevent its solidification, and glucose tends to foam during autoclaving. As such, glucose should be separated between the two bottles to prevent boil-over during autoclaving or Bottle 1 and Bottle 2 should include sufficient headspace. The final concentration of glucose in the media was 21%.

TABLE 3
EXPERIMENTAL FORMULATION
OF LIQUID MEDIA BOTTLES

| Component | Bottle 1 | Bottle 2 | Quantity | |
|---|---|---|---|---|
| Formic Acid | 0.20% v/v | — | 2.0 | mL |
| Acetic Acid | 0.25% v/v | — | 2.5 | mL |
| 5 M Sodium Hydroxide | to pH 3.6-3.7 | — | — | |
| Bromocresol Green | 0.0025% w/v | — | 25 | mL |
| Glucose | 10% w/v | — | 100 | g |
| Peptone | — | 1.0% w/v | 10 | g |
| Yeast Extract | — | 0.5% w/v | 5 | g |
| Agar | — | 2.0% w/v | 20 | g |
| Glucose | — | 11.0% w/v | 110 | g |

| | | | |
|---|---|---|---|
| Water | to 500 ml | to 500 ml | — |

While illustrative embodiments have been illustrated and described, it will be appreciated that various changes can be made therein without departing from the spirit and scope of the invention. The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

1. A growth medium selective for Zygosaccharomyces bailii, comprising:
a nutrient base;
about 1%-25% w/v of a sugar;
about 0.1%43% v/v formic acid; and
about 0.2%45% v/v acetic acid;
wherein the growth medium is characterized by a final pH of between about 2.5 to about 4.

2. The growth medium of claim 1, wherein the nutrient base comprises peptone or yeast extract.

3. The growth medium of claim 2, wherein the nutrient base comprises peptone at a final medium concentration of about 0.5%-2% w/v, yeast extract at a final medium concentration of about 0.1%4% w/v, and a final medium concentration of about 0.6%-3% w/v of the nutrient base in the growth medium.

4. The growth medium of claim 1, wherein the concentration of the sugar in the growth medium is about 5%-25% w/v, about 7%-25% w/v, about 10%-25% w/v, about 12%-25% w/v, about 15%-25% w/v, about 17%-25% w/v, about 20%-25% w/v, about 5%-23% w/v, about 7%-23% w/v, about 10%-23% w/v, about 12%-23% w/v, about 15%-23% w/v, about 17%-23% w/v, about 20%-23% w/v, about 7%-21% w/v, about 10%-21% w/v, about 12%-21% w/v, about 15%-21% w/v, about 17%-21% w/v, or about 20%-21% w/v.

5. The growth medium of claim 1, wherein the concentration of the sugar in the growth medium is about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, or about 25% w/v.

6. The growth medium of claim 1, wherein the concentration of formic acid is about 0.12%-0.3% v/v, about 0.15%-0.3% v/v, about 0.17%-0.3% v/v, about 0.2%-0.3% v/v, about 0.22%-0.3% v/v, about 0.25%-0.3% v/v, about 0.12%-0.27% v/v, about 0.15%-0.27% v/v, about 0.17%-0.27% v/v, about 0.2%-0.27% v/v, about 0.22%-0.27% v/v, about 0.25%-0.27% v/v, about 0.12%-0.25% v/v, about 0.15%-0.25% v/v, about 0.17%-0.25% v/v, about 0.2%-0.25% v/v, about 0.12%-0.22% v/v, about 0.15%-0.22% v/v, about 0.17%-0.22% v/v, or about 0.2%-0.22% v/v.

7. The growth medium of claim 1, wherein the concentration of formic acid is about 0.12%, about 0.15%, about 0.17%, about 0.2%, about 0.22%, about 0.25%, about 0.27%, or about 0.3%.

8. The growth medium of claim 1, wherein the concentration of acetic acid is about 0.22%-0.5% v/v, about 0.25%-0.5% v/v, about 0.27%-0.5% v/v, about 0.3%-0.5% v/v, about 0.32%-0.5% v/v, about 0.35%-0.5% v/v, about 0.37%-0.5% v/v, about 0.4-0.5% v/v, about 0.42%-0.5% v/v, about 0.45%-0.5% v/v, about 0.47%-0.5% v/v, about 0.22%-0.45% v/v, about 0.25%-0.45% v/v, about 0.27%-0.45% v/v, about 0.3%-0.45% v/v, about 0.32%-0.45% v/v, about 0.35%-0.45% v/v, about 0.37%-0.45% v/v, about 0.4%-0.45% v/v, about 0.42%-0.45% v/v, about 0.22%-0.4% v/v, about 0.25%-0.4% v/v, about 0.27%-0.4% v/v, about 0.3%-0.4% v/v, about 0.32%-0.4% v/v, about 0.35%-0.4% v/v, about 0.37%-0.4% v/v, about 0.22%-0.35% v/v, about 0.25%-0.35% v/v, about 0.27%-0.35% v/v, about 3%-3.5% v/v, about 3.2%-3.5% v/v, about 0.22%-0.3% v/v, about 0.25%-0.3% v/v, about 0.27%-0.3% v/v, about 0.22%-0.27% v/v, about 0.25%-0.27% v/v, or about 0.27%-0.3% v/v.

9. The growth medium of claim 1, wherein the concentration of acetic acid is about 0.2% v/v, about 0.25% v/v, about 0.27% v/v, about 0.3% v/v, about 0.32% v/v, about 0.35% v/v, about 0.37% v/v, about 0.4% v/v, about 0.42% v/v, about 0.45% v/v, about 0.47% v/v, or about 0.5% v/v.

10. The growth medium of claim 1, wherein the final pH is between about 2.7 and 4, between about 3 and 4, between about 3.2 and 4, between about 3.5 and 4, between about 3.7 and 4, between about 2.7 and 3.7, between about 3 and 3.7, between about 3.2 and 3.7, between about 3.5 and 3.7, between about 2.7 and 3.5, between about 3 and 3.5, between about 3.2 and 3.5, between about 2.7 and 3.2, or between about 3 and 3.2.

11. The growth medium of claim 1, wherein the final pH is about 2.5, about 2.6, about 2.7, about 2.8, about 2.9, about 3, about 3.1, about 3.2, about 3.3, about 3.4, about 3.5, about 3.6, about 3.7, about 3.8, about 3.9, or about 4.

12. The growth medium of claim 1, comprising:
about 0.5%-1.5% w/v peptone;
about 0.25%-0.75% w/v yeast extract;
about 1.5%-2.5% w/v agar;
about 10%-25% w/v the sugar;
about 0.15%-0.25% v/v formic acid;
about 0.22%-0.45% v/v acetic acid; and
wherein the growth medium is characterized by a final pH of between about 3 to about 4.

13. The growth medium of claim 12, comprising:
about 1% w/v peptone;
about 0.5% w/v yeast extract;
about 2% w/v agar;
about 15%-22% w/v the sugar;
about 0.2% v/v formic acid;
about 0.25% v/v acetic acid; and
wherein the growth medium is characterized by a final pH of between about 3 to about 4.

14. The growth medium of claim 1, wherein the growth medium further comprises bromocresol green at a concentration of about 0.001% w/v to about 0.005% w/v.

15. The growth medium of claim 1, wherein the sugar comprises glucose or fructose.

16. A method of making a growth medium for Zygosaccharomyces bailii, the method comprising
autoclaving a first liquid medium comprising formic acid, acetic acid, between about 30% and 70% of a total amount of a sugar in the growth medium, and water;
autoclaving a second liquid medium comprising peptone, yeast extract, agar, between about 30% and 70% of the total amount of the sugar in the growth medium, and water;
combining the first liquid medium and second liquid medium, wherein the combination of the first liquid medium and second liquid medium comprises the growth medium and includes the total amount of the sugar in the growth medium; and
solidifying the growth medium to provide a solid growth medium,
wherein the solid growth medium comprises:
about 0.5%-1.5% w/v peptone;
about 0.25%-0.75% w/v yeast extract;
about 1.5%-2.5% w/v agar;
about 10%-25% w/v of the sugar comprising glucose or fructose;
about 0.15%-0.25% v/v formic acid;
about 0.22%-0.45% v/v acetic acid; and
wherein the solid growth medium is characterized by a pH of between about 3 to about 4.

17. A method of selectively cultivating Zygosaccharomyces bailii, comprising:
inoculating a selective growth medium with a sample ; and
incubating the inoculated medium for at least 36 hours, wherein the growth medium comprises:
a nutrient base comprising peptone and yeast extract;
about 10%-25% w/v of a sugar comprising glucose or fructose;
about 0.15%-0.25% v/v formic acid;
about 0.22%-0.45% v/v acetic acid; and
is characterized by a pH of between about 3 to about 4.

18. The method of claim 17, further comprising incubating the inoculated medium at a temperature from about 25° C. to about 30° C.

19. The method of claim 17, wherein the sample comprises or is derived from food, food waste, beverages, beverage waste, liquids, agricultural products, or agricultural byproducts.

20. The method of claim 17, wherein the sample is a test sample created from a surface that has contact with food, food waste, beverages, beverage waste, liquids, agricultural products, or agricultural byproducts.
